(19) United States
(12) Patent Application Publication
(10) Pub. No.: US 2011/0110996 A1
(43) Pub. Date: May 12, 2011

(54) LAYER COMPOSITE, SUITABLE AS A WOUND DRESSING, COMPRISING A POLYURETHANE FOAM LAYER, AN ABSORBER LAYER AND A COVER LAYER

(75) Inventors: Jan Schoenberger, Haan (DE); Michael Mager, Leverkusen (DE)

(73) Assignee: BAYER MATERIALSCIENCE AG, Leverkusen (DE)

(21) Appl. No.: 13/001,927

(22) PCT Filed: Jun. 20, 2009
(86) PCT No.: PCT/EP2009/004476

§ 371 Date: Dec. 29, 2010

(30) Foreign Application Priority Data
Jul. 4, 2008 (EP) 08159768.4

(51) Int. Cl. A61K 8/02; A61K 9/00; A61P 17/02; A61Q 19/00

(52) U.S. Cl. 424/401; 424/400

(57) ABSTRACT

Layered composite useful as wound dressing, comprising a base layer, an absorbent layer atop the base layer, and also a covering layer, wherein the covering layer is bonded to both the base layer and the absorbent layer and wherein the base layer comprises a polyurethane foam obtained by a composition comprising an aqueous, anionically hydrophilicized polyurethane dispersion that has been frothed and dried.

The present invention relates to a layered composite which is useful as a wound dressing. The invention further relates to a process for producing such a layered composite and to its use as a wound dressing.

In the management of open wounds and particularly of chronic open wounds such as ulcers, the excess moisture produced by the wound should be absorbed during the exudative phase of wound healing. Wound infections could otherwise result due to a blockage of exudate. Superabsorbent polymers are very effective means for absorbing moisture. However, superabsorbent polymers cannot be applied directly to the skin or even to the open wound. There is consequently a need for an interlayer between the wound and the absorbent. Furthermore, the absorbent is generally covered by a further layer to obtain a wound plaster.

WO 2007/115696 discloses a process for producing polyurethane foams for wound treatment wherein a composition comprising a polyurethane dispersion and specific coagulants is frothed and dried. The polyurethane dispersions are obtainable for example by preparing isocyanate-functional prepolymers from organic polyisocyanates and polymeric polyols having number average molecular weights of 400 g/mol to 8000 g/mol and OH functionalities of 1.5 to 6 and also optionally with hydroxyl-functional compounds having molecular weights of 62 g/mol to 399 g/mol and optionally isocyanate-reactive, anionic or potentially anionic and optionally nonionic hydrophilicizing agents. The free NCO groups of the prepolymer are then optionally reacted in whole or in part with amino-functional compounds having molecular weights of 32 g/mol to 400 g/mol and also with amino-functional, anionic or potentially anionic hydrophilicizing agents with chain extension. The prepolymers are dispersed in water before, during or after the step of chain extension. Any potential ionic groups present are converted into the ionic form by partial or complete reaction with a neutralizing agent.

EP 0 760 743 discloses layered articles for absorbing water and aqueous fluid which consist of at least one plastics foam and/or latex foam layer and also particulate superabsorbent addition polymers and which contain the superabsorbent, on, between or under the foamed plastics and/or latex layer, in a quantitatively and/or geometrically predetermined and fixed planar arrangement in a quantitative ratio ranging from 1:500 to 50:1 for plastics and/or latex foam to superabsorbent. Plastics/latex foam may contain fillers, pigments and/or synthetic fibres. The layered articles have enhanced absorbency for water and aqueous fluids, particularly under a confining pressure. They are obtained by the foam being distributed in planar form and the superabsorbent being applied in the predetermined quantitative ratio, with or without use of a template, and fixed by heat treatment.

Such layered articles are used in hygiene products, as components in natural or artificial soils, as an insulating material for pipes and lines, particularly cables, and built structures, as liquid-imbibing and -storing component in packaging materials, and also as a component in apparel pieces.

WO 2001/60422 discloses medical articles such as wound dressings for example. In one embodiment, the medical article comprises a backing, an absorbent foam and a fibrous adhesive between the backing and the absorbent foam, the backing comprising a liquid-impervious, moisture-vapour permeable polymeric film. In another embodiment, the medical article comprises a backing, an absorbent, substantially nonswellable foam and an adhesive disposed therebetween. In yet another embodiment, the medical article comprises a backing, a foam and a fibrous adhesive disposed therebetween.

WO 2002/43784 discloses a layer for personal care products comprising elastic polymers which are extruded and are made superabsorbent by crosslinking Such a layer can be used in personal care products such as diapers, training pants, incontinence apparel and feminine hygiene products.

WO 2006/089551 discloses a wound dressing comprising a backing layer and a skin-facing layer and an absorbent pad, wherein the absorbent pad is sandwiched between the backing layer and the skin-facing layer, and the two layers constitute an envelope, and the absorbent pad has an expansion of surface area, when fully expanded, of at least 10%. The surface area of the envelope is at least 10% larger than the surface area of the non-expanded absorbent pad. The envelope provides space for the absorbent pad to expand into without the absorbent pad having to be bent or folded.

US 2006/211781 A1 discloses layer-shaped, multilayered froth laminates consisting of aqueous olefin polymers and useful for absorbing water and aqueous fluids. They are made by distributing the froth in sheet form. The dried froth then serves as substrate for the next layer of froth. This method permits a sandwich construction of froth/substrate/froth/substrate wherein the substrate may comprise a froth other than the first. Neither polyurethane dispersions nor the use of super-absorbent polymers are disclosed.

Layer-shaped wound dressings comprising an absorbent layer hitherto had to be manufactured, when layers in foam form were used, by using an adhesive to ensure adequate adherence of the absorbent layer or of a covering layer to the foam layer. This is disadvantageous since, on the one hand, an additional operation was needed, which often had to be carried out by hand, and, on the other, the introduction of the adhesive into the bandage introduces an additional risk of unwanted effects occurring.

There is therefore a need for improved or at least alternative wound dressings which can be produced using a smaller number of fabrication steps and using fewer materials. More particularly, it would be desirable if no adhesive had to be used to bond layers together within the wound dressing.

The invention therefore proposes a layered composite useful as wound dressing, comprising a base layer, an absorbent layer atop the base layer, and also a covering layer, wherein the covering layer is bonded to both the base layer and the absorbent layer and wherein the base layer comprises a polyurethane foam obtained by a composition comprising an aqueous, anionically hydrophilicized polyurethane dispersion (I) being frothed and dried.

The layered composite of the invention is likewise useful as an incontinence article or as a cosmetic article as well as other uses.

The layered composite of the invention can be regarded as an island dressing, in which case the absorbent layer is enclosed by the base layer and the covering layer. The covering layer is therefore in direct contact with the base layer in those areas where it is not in contact with the absorbent layer.

It is contemplated that the base layer comprises a foam which is obtainable from a frothed polyurethane dispersion. This base layer is placed on the wound to be covered. Advantageously, this foam has a microporous, at least partly open-pore structure comprising intercommunicating cells.

The polyurethane dispersion (I) comprises polyurethanes prepared by reacting free isocyanate groups as a whole or in part with anionic or potentially anionic hydrophilicizing agents. Such hydrophilicizing agents are compounds which have isocyanate-reactive functional groups such as amino, hydroxyl or thiol groups as well as acid or acid anion groups such as carboxylate, sulphonate or phosphonate groups.

The absorbent layer comprises a material capable of binding water or other liquids. The absorbent layer differs from the base layer. For example, the absorbent layer may comprise superabsorbent polymers (SAPs), also known as superabsorbents. Such superabsorbents are materials having the ability to absorb and retain an amount of water equivalent to many times their own weight, even under moderate pressure. Their Centrifuge Retention Capacity (CRC) is generally at least 5 g/g, preferably at least 10 g/g and more preferably at least 15 g/g.

Centrifuge Retention Capacity is determined by following the eponymous test method No. 441.2-02 recommended by EDANA (European Disposables and Nonwovens Association, Avenue Eugene Plasky 157, 1030 Brussels, Belgium) and available from there.

Superabsorbents are particularly polymers of (co)polymerized hydrophilic monomers, graft (co)polymers of one or more hydrophilic monomers on a suitable grafting base, crosslinked ethers of cellulose or of starch, crosslinked carboxymethylcellulose, partially crosslinked polyalkylene oxide or natural products which are swellable in aqueous fluids, such as guar derivatives for example, and also preferably water-absorbing polymers based on partially neutralized acrylic acid. A superabsorbent can also be a mixture of chemically different individual superabsorbents.

The covering layer of the layered composite of the invention is initially not fixed with regard to the choice of material of construction. The covering layer is advantageously elastic in order that any increase in volume due to swelling of the absorbent layer may be compensated.

More particularly, useful materials for the covering layer include such foams, films or foam-films as are fabricated from elastomeric polymers based on polyurethane, polyethylene, polypropylene, polyvinyl chloride, polystyrene, polyether, polyester, polyamide, polycarbonate, polycarboxylic acids such as polyacrylic acids, polymethacrylic acids, polymaleic acids, also polyvinyl acetate, polyvinyl alcohol, cellulose ester and/or mixtures thereof. But it is also possible to use wovens and nonwovens based on natural fibres, such as cellulose, cotton or linen, and also plastic-coated wovens and nonwovens.

It will further be found particularly advantageous when films have thicknesses in the range from ≧5 μm to ≦80 μm, in particular from ≅5 μm to ≦60 μm and more preferably from ≧10 m to ≦30 μm, and a breaking extension of above 450%.

A layered composite according to the invention may utilize particularly such polymeric films as have a high water vapour permeability. Films particularly suitable for this purpose are fabricated from polyurethane, polyether urethane, polyester urethane, polyether-polyamide copolymers, polyacrylate or polymethacrylate. Particular preference for use as polymeric film is given to polyurethane film, polyester polyurethane film or polyether polyurethane film. Very particular preference is given to such films as have a thickness of ≧5 μm to ≦80 μm, particularly of ≧5 μm to ≦60 μm and more preferably of ≧10 μm to ≦30 μm.

Moisture emerging from the wound in liquid form or in vapour form is transported away from the wound through the open-pore network of the polyurethane foam of the base layer, and can be imbibed by the absorbent layer.

We have found that a layered composite according to the present invention is capable of imbibing moisture in the absorbent layer, and of swelling, without deterioration in the performance of the bond of adherence between the base layer and the covering layer. In other words, the bond between the base layer and the covering layer is so stable that there is no need for additional adhesive between the base layer and the covering layer.

The layered composite according to the present invention thus provides a wound dressing which, owing to the elimination of the special adhering together of base layer and covering layer, is simpler to manufacture. The elimination of a layer of adhesive further does away with a source of potential failure in the presence of moisture. The use of the polyurethane foam for the base layer is particularly advantageous since the foam combines good vapour permeability with sufficient adhesiveness of its own.

In one embodiment of the layered composite according to the invention, the composition from which the polyurethane foam of the base layer is obtained further comprises admixtures selected from the group comprising fatty acid amides, sulphosuccinamides, hydrocarbonsulphonates, hydrocarbyl sulphates, fatty acid salts, alkylpolyglycosides and/or ethylene oxide-propylene oxide block copolymers.

Such admixtures can act as foam formers and/or foam stabilizers. The lipophilic radical in the fatty acid amides, sulphosuccinamides, hydrocarbonsulphonates, hydrocarbyl sulphates or fatty acid salts preferably comprises ≧12 to ≦24 carbon atoms. Suitable alkylpolyglycosides are obtainable for example by reaction of comparatively long-chain monoalcohols (≧4 to ≦22 carbon atoms in the alkyl radical) with mono-, di- or polysaccharides. Also suitable are alkylbenzosulphonates or alkylbenzene sulphates having ≧14 to ≦24 carbon atoms in the hydrocarbyl radical.

The fatty acid amides are preferably those based on mono- or di-(C₂/C₃-alkanol)amines The fatty acid salts can be for example alkali metal salts, amine salts or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coco fatty acid, tallow fatty acid, soya fatty acid and hydrogenation products thereof

Exemplarily useful foam stabilizers are mixtures of sulphosuccinamides and ammonium stearates, the ammonium stearate content being preferably ≧20% by weight to ≦60% by weight, more preferably ≧30% by weight to ≦50% by weight, and the sulphosuccinamide content being preferably ≧40% by weight, to ≦80% by weight, more preferably ≧50% by weight to ≦70% by weight.

Further exemplarily useful foam stabilizers are mixtures of fatty alcohol-polyglycosides and ammonium stearates, the ammonium stearate content being preferably ≧20% by weight to ≦60% by weight and more preferably ≧30% by weight to ≦50% by weight and the fatty alcohol-polyglycoside content being preferably ≧40% by weight to ≦80% by weight and more preferably ≧50% by weight to ≦70% by weight.

The ethylene oxide/propylene oxide block copolymers comprise addition products of ethylene oxide and propylene oxide onto OH- or NH-functional starter molecules.

Useful starter molecules include in principle inter alia water, polyethylene glycols, polypropylene glycols, glycerol, trimethylolpropane, pentaerythritol, ethylenediamine, tolylenediamine, sorbitol, sucrose and mixtures thereof

Preference is given to using di- or trifunctional compounds of the aforementioned kind as starters. Particular preference is given to polyethylene glycol or polypropylene glycol.

By varying the amount of alkylene oxide in each case and the number of ethylene oxide (EO) and propylene oxide (PO) blocks it is possible to obtain block copolymers of various kinds.

It is also possible in principle for copolymers constructed strictly blockwise from ethylene oxide or propylene oxide to also include individual mixed blocks of EO and PO.

Such mixed blocks are obtained on using mixtures of EO and PO in the polyaddition reaction so that, in relation to this block, a random distribution of EO and PO results in this block.

The ethylene oxide content of the EO/PO block copolymers used according to the invention is preferably ≧5% by weight, more preferably ≧20% by weight and most preferably ≧40% by weight, based on the sum total of the ethylene oxide and propylene oxide units present in the copolymer.

The ethylene oxide content of the EO/PO block copolymers used according to the invention is preferably ≦95% by weight, more preferably ≦90% by weight and most preferably ≦85% by weight based on the sum total of the ethylene oxide and propylene oxide units present in the copolymer.

The number average molecular weight of the EO/PO block copolymers used according to the invention is preferably ≧1000 g/mol, more preferably ≦2000 g/mol and most preferably ≧5000 g/mol.

The number average molecular weight of the EO/PO block copolymers used according to the invention is preferably ≦10 000 g/mol, more preferably ≦9500 g/mol and most preferably ≦9000 g/mol.

One advantageous aspect of using the EO/PO block copolymers is that the foam obtained has a lower hydrophobicity than when other stabilizers are used. The imbibition behaviour for liquids can be favourably influenced as a result. Moreover, non-zytotoxic foams are obtained when EO/PO block copolymers are used, in contradistinction to other stabilizers.

It is possible for the ethylene oxide/propylene oxide block copolymers to have a structure conforming to the general formula (1):

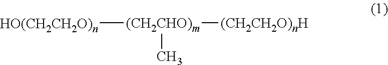

where n is in the range from ≧2 to ≦200, preferably from ≧60 to ≦180 and more preferably from ≧130 to ≦160 and m is in the range from ≧10 to ≦60, preferably from ≧25 to ≦45 and more preferably from ≧25 to ≦35.

EO/PO block copolymers of the aforementioned kind are particularly preferred in that they have a hydrophilic-lipophilic balance (HLB) of ≧4, more preferably of ≧8 and most preferably of ≧14. The HLB value computes according to the formula HLB=20·Mh/M, where Mh is the number average molar mass of the hydrophilic moiety, formed from ethylene oxide, and M is the number average molar mass of the overall molecule (Griffin, W. C.: Classification of surface active agents by HLB, J. Soc. Cosmet. Chem. 1, 1949). However, the HLB value is ≦19 and preferably ≦18.

In one embodiment of the layered composite of the invention, the aqueous anionically hydrophilicized polyurethane dispersion (I) is obtainable by
A) providing isocyanate-functional prepolymers obtainable from a reaction mixture comprising
   A1) organic polyisocyanates and
   A2) polymeric polyols having number average molecular weights of ≧400 g/mol to ≦8000 g/mol and OH functionalities of ≧1.5 to ≦6
      and subsequently
B) reacting the free NCO groups of the prepolymers in whole or in part with
   B1) isocyanate-reactive anionic or potentially anionic hydrophilicizing agents
      with chain extension and dispersing the prepolymers in water before, during or after step B), wherein potentially anionic groups still present in the reaction mixture are converted into their ionic form by partial or complete reaction with a neutralizing agent.

Preferred aqueous, anionic polyurethane dispersions (I) have a low degree of hydrophilic anionic groups, preferably in the range from ≧0.1 to ≦15 milliequivalents per 100 g of solid resin.

To achieve good sedimentation stability, the number average particle size of the specific polyurethane dispersions is preferably ≦750 nm and more preferably ≦500 nm, determined by means of laser correlation spectroscopy.

The ratio of NCO groups of compounds of component A1) to NCO-reactive groups such as amino, hydroxyl or thiol groups of compounds of components A2) to A4) is ≧1.05 to ≦3.5, preferably ≧1.2 to ≦3.0 and more preferably ≧1.3 to ≦2.5 to prepare the NCO-functional prepolymer.

The amino-functional compounds in stage B) are used in such an amount that the equivalent ratio of isocyanate-reactive amino groups of these compounds to the free isocyanate groups of the prepolymer is ≧40% to ≦150%, preferably between ≧50% and ≦125% and more preferably between ≧60% and ≦120%.

Suitable polyisocyanates for component A1) are aromatic, araliphatic, aliphatic or cycloaliphatic polyisocyanates of an NCO functionality of ≧2.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any desired isomer content, 1,4-cyclohexylene diisocyanate, 1,4-phenylene diisocyanate, 2,4- and/or 2,6-tolylene diisocyanate (TDT), 1,5-naphthylene diisocyanate, 2,2′- and/or 2,4′- and/or 4,4′-diphenylmethane diisocyanate, 1,3- and/or 1,4-bis-(2-isocyanatoprop-2-yl)benzene (TMXDI), 1,3-bis(isocyanatomethyl)benzene (XDI), and also alkyl 2,6-diisocyanatohexanoates (lysine diisocyanates) having C₁-C₈-alkyl groups.

As well as the aforementioned polyisocyanates, it is also possible to use, proportionally, modified diisocyanates of uretdione, isocyanurate, urethane, allophanate, biuret, iminooxadiazinedione and/or oxadiazinetrione structure and also non-modified polyisocyanate having more than 2 NCO groups per molecule, for example 4-isocyanatomethyl-1,8-octane diisocyanate (nonane triisocyanate) or triphenylmethane 4,4′,4″-triisocyanate.

Preferably, the polyisocyanates or polyisocyanate mixtures of the aforementioned kind have exclusively aliphatically and/or cycloaliphatically attached isocyanate groups and an average NCO functionality of ≧2 to ≦4, preferably ≧2 to ≦2.6 and more preferably ≧2 to ≦2.4 for the mixture.

It is particularly preferable for A1) to utilize 1,6-hexamethylene diisocyanate, isophorone diisocyanate, the isomeric bis(4,4′-isocyanatocyclohexyl)methanes, and also mixtures thereof.

A2) utilizes polymeric polyols having a number average molecular weight Mₙ of ≧400 to ≦8000 g/mol, preferably from ≧400 to ≦6000 g/mol and more preferably from ≧600 to ≦3000 g/mol. These preferably have an OH functionality of ≧1.5 to ≦6, more preferably of ≧1.8 to ≦3 and most preferably of ≧1.9 to ≦2.1.

Such polymeric polyols include for example polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These can be used in A2) individually or in any desired mixtures with one another.

Such polyester polyols include polycondensates formed from di- and also optionally tri- and tetraols and di- and also optionally tri- and tetracarboxylic acids or hydroxy carboxylic acids or lactones. Instead of the free polycarboxylic acids it is also possible to use the corresponding polycarboxylic anhydrides or corresponding polycarboxylic esters of lower alcohols for preparing the polyesters.

Examples of suitable diols are ethylene glycol, butylene glycol, diethylene glycol, triethylene glycol, polyalkylene glycols such as polyethylene glycol, also 1,2-propanediol, 1,3-propanediol, butanediol(1,3), butanediol(1,4), hexanediol(1,6) and isomers, neopentyl glycol or neopentyl glycol hydroxypivalate, of which hexanediol(1,6) and isomers, neopentyl glycol and neopentyl glycol hydroxypivalate are preferred. Besides these it is also possible to use polyols such as trimethylolpropane, glycerol, erythritol, pentaerythritol, trimethylolbenzene or trishydroxyethyl isocyanurate.

Useful dicarboxylic acids include phthalic acid, isophthalic acid, terephthalic acid, tetra-hydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethyl glutaric acid and/or 2,2-dimethylsuccinic acid. The corresponding anhydrides can also be used as a source of an acid.

When the average functionality of the polyol to be esterified is 2, mono carboxylic acids, such as benzoic acid and hexanecarboxylic acid can be used as well in addition.

Preferred acids are aliphatic or aromatic acids of the aforementioned kind. Adipic acid, isophthalic acid and optionally trimellitic acid are particularly preferred.

Hydroxy carboxylic acids useful as reaction participants in the preparation of a polyester polyol having terminal hydroxyl groups include for example hydroxycaproic acid, hydroxybutyric acid, hydroxydecanoic acid, hydroxystearic acid and the like. Suitable lactones include caprolactone, butyrolactone and homologues. Caprolactone is preferred.

A2) may likewise utilize hydroxyl-containing polycarbonates, preferably polycarbonate diols, having number average molecular weights Mₙ of ≧400 to ≦8000 g/mol and preferably in the range from ≧600 to ≦3000 g/mol. These are obtainable by reaction of carbonic acid derivatives, such as diphenyl carbonate, dimethyl carbonate or phosgene, with polyols, preferably diols.

Examples of such diols are ethylene glycol, 1,2-propanediol, 1,3-propanediol, 1,3-butanediol, 1,4-butanediol, 1,6-hexanediol, 1,8-octanediol, neopentyl glycol, 1,4-bishydroxymethyl-cyclohexane, 2-methyl-1,3-propanediol, 2,2,4-trimethyl-1,3-pentanediol, dipropylene glycol, polypropylene glycols, dibutylene glycol, polybutylene glycols, bisphenol A and lactone-modified diols of the aforementioned kind.

The polycarbonate diol preferably contains ≧40% to ≦100% by weight of hexanediol, preference being given to 1,6-hexanediol and/or hexanediol derivatives. Such hexanediol derivatives are based on hexanediol and have ester or ether groups as well as terminal OH groups. Such derivatives are obtainable by reaction of hexanediol with excess caprolactone or by etherification of hexanediol with itself to form di- or trihexylene glycol.

In lieu of or in addition to pure polycarbonate diols, polyether-polycarbonate diols can also be used in A2).

Hydroxyl-containing polycarbonates preferably have a linear construction.

A2) may likewise utilize polyether polyols.

Useful for example are polytetramethylene glycol polyethers as are obtainable by polymerization of tetrahydrofuran by means of cationic ring opening.

Useful polyether polyols likewise include the addition products of styrene oxide, ethylene oxide, propylene oxide, butylene oxide and/or epichlorohydrin onto di- or polyfunctional starter molecules. Polyether polyols based on the at least proportional addition of ethylene oxide onto di- or polyfunctional starter molecules can also be used as component A4) (nonionic hydrophilicizing agents).

Useful starter molecules include for example water, butyl diglycol, glycerol, diethylene glycol, trimethylolpropane, propylene glycol, sorbitol, ethylenediamine, triethanolamine, or 1,4-butanediol. Preferred starter molecules are water, ethylene glycol, propylene glycol, 1,4-butanediol, diethylene glycol and butyl diglycol.

Particularly preferred embodiments of the polyurethane dispersions (I) contain as component A2) a mixture of polycarbonate polyols and polytetramethylene glycol polyols, the proportion of polycarbonate polyols in this mixture being ≧20% to ≦80% by weight and the proportion of polytetramethylene glycol polyols in this mixture being ≧20% to ≦80% by weight. Preference is given to a proportion of ≧30% to ≦75% by weight for polytetramethylene glycol polyols and to a proportion of ≧25% to ≦70% by weight for polycarbonate polyols. Particular preference is given to a proportion of ≧35% to ≦70% by weight for polytetramethylene glycol polyols and to a proportion of ≧30% to ≦65% by weight for polycarbonate polyols, each subject to the proviso that the sum total of the weight percentages for the polycarbonate and polytetramethylene glycol polyols is 100% by weight and the proportion of component A2) which is accounted for by the sum total of the polycarbonate polyols and polytetramethylene glycol polyether polyols is ≧50% by weight, preferably ≧60% by weight and more preferably ≧70% by weight.

An isocyanate-reactive anionic or potential anionic hydrophilicizing agent of component B1) is any compound which has at least one isocyanate-reactive group such as an amino, hydroxyl or thiol group and also at least one functionality such as for example —COO⁻M⁺, —SO₃⁻M³⁰ , —PO(O⁻M⁺)₂ where M⁺ is for example a metal cation, H⁺, NH₄⁺, NHR₃⁺, where R in each occurrence may be C₁-C₁₂-alkyl, C₅-C₆-cycloalkyl and/or C₂-C₄-hydroxyalkyl, which functionality on interaction with aqueous media enters a pH-dependent dissociative equilibrium and thereby can have a negative or neutral charge.

The isocyanate-reactive anionic or potentially anionic hydrophilicizing agents are preferably isocyanate-reactive amino-functional anionic or potentially anionic hydrophilicizing agents.

Useful anionically or potentially anionically hydrophilicizing compounds are mono- and diamino carboxylic acids, mono- and diamino sulphonic acids and also mono- and diamino phosphonic acids and their salts. Examples of such anionic or potentially anionic hydrophilicizing agents are N-(2-aminoethyl)-β-alanine, 2-(2-aminoethylamino)ethanesulphonic acid, ethylenediaminepropylsulphonic acid, ethylenediaminebutylsulphonic acid, 1,2- or 1,3-propylenediamine-β-ethylsulphonic acid, glycine, alanine, taurine, lysine, 3,5-diaminobenzoic acid and the addition product of IPDA and acrylic acid (EP-A 0 916 647, Example 1). It is further possible to use cyclohexylaminopropanesulphonic acid (CAPS) from WO-A 01/88006 as anionic or potentially anionic hydrophilicizing agent.

Preferred anionic or potentially anionic hydrophilicizing agents for component B1) are those of the aforementioned kind that have carboxylate or carboxyl groups and/or sulphonate groups, such as the salts of N-(2-aminoethyl)-β-alanine, of 2-(2-aminoethylamino)ethanesulphonic acid or of the addition product of IPDA and acrylic acid (EP-A 0 916 647, Example 1).

Mixtures of anionic or potentially anionic hydrophilicizing agents and nonionic hydrophilicizing agents can also be used.

In a further embodiment of the layered composite of the present invention, the reaction mixture in step A) further comprises:
A3) hydroxyl-functional compounds having molecular weights of ≧62 g/mol to ≦399 g/mol.

The compounds of component A3) have molecular weights of ≧62 to ≦399 g/mol.

A3) may utilize polyols of the specified molecular weight range with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxy-phenyl)propane), hydrogenated bisphenol A, (2,2-bis(4-hydroxycyclohexyl)propane), trimethylol-propane, glycerol, pentaerythritol and also any desired mixtures thereof with one another.

Also suitable are ester diols of the specified molecular weight range such as α-hydroxybutyl-ε-hydroxycaproic acid ester, ω-hydroxyhexyl-γ-hydroxybutyric acid ester, β-hydroxyethyl adipate or bis(β-hydroxyethyl)terephthalate.

A3) may further utilize monofunctional isocyanate-reactive hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferred compounds for component A3) are 1,6-hexanediol, 1,4-butanediol, neopentyl glycol and trimethylolpropane.

In a further embodiment of the layered composite of the invention, the reaction mixture in step A) further comprises:
A4) isocyanate-reactive anionic, potentially anionic and/or nonionic hydrophilicizing agents.

An anionically or potentially anionically hydrophilicizing compound for component A4) is any compound which has at least one isocyanate-reactive group such as a hydroxyl group and also at least one functionality such as for example —COO⁻M⁺, —SO₃⁻M⁺, —PO(O⁻M⁺)₂ where M⁺ is for example a metal cation, H⁺, NH₄⁺, NHR₃⁺, where R in each occurrence may be C₁-C₁₂ -alkyl, C₅-C₆-cycloalkyl and/or C₂-C₄-hydroxyalkyl, which functionality enters on interaction with aqueous media a pH-dependent dissociative equilibrium and thereby can have a negative or neutral charge. Useful anionically or potentially anionically hydrophilicizing compounds include for example mono- and dihydroxy carboxylic acids, mono- and dihydroxy sulphonic acids and also mono- and dihydroxy phosphonic acids and their salts. Examples of such anionic or potentially anionic hydrophilicizing agents are dimethylolpropionic acid, dimethylolbutyric acid, hydroxypivalic acid, malic acid, citric acid, glycolic acid, lactic acid and the propoxylated adduct formed from 2-butenediol and NaHSO₃ as described in DE-A 2 446 440, page 5-9, formula I-III. Preferred anionic or potentially anionic hydrophilicizing agents for component A4) are those of the aforementioned kind that have carboxylate or carboxyl groups and/or sulphonate groups.

Particularly preferred anionic or potentially anionic hydrophilicizing agents are those that contain carboxylate or carboxyl groups as ionic or potentially ionic groups, such as dimethylolpropionic acid, dimethylolbutyric acid and hydroxypivalic acid and/or salts thereof.

Useful nonionically hydrophilicizing compounds for component A4) include for example polyoxyalkylene ethers which contain at least one hydroxyl or amino group, preferably at least one hydroxyl group. Examples thereof are the monohydroxyl-functional polyalkylene oxide polyether alcohols containing on average ≧5 to ≦70 and preferably ≧7 to ≦55 ethylene oxide units per molecule and obtainable by alkoxylation of suitable starter molecules. These are either pure polyethylene oxide ethers or mixed polyalkylene oxide ethers, containing ≧30 mol % and preferably ≧40 mol % of ethylene oxide units, based on all alkylene oxide units present.

Preferred polyethylene oxide ethers of the aforementioned kind are monofunctional mixed polyalkylene oxide polyethers having ≧40 mol % to ≦100 mol % of ethylene oxide units and ≧0 mol % to ≦60 mol % of propylene oxide units.

Preferred nonionically hydrophilicizing compounds for component A4) include those of the aforementioned kind that are block (co)polymers prepared by blockwise addition of alkylene oxides onto suitable starters.

Useful starter molecules for such nonionic hydrophilicizing agents include saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or oleic alcohol, aromatic alcohols such as phenol, the isomeric cresols or methoxyphenols, araliphatic alcohols such as benzyl alcohol, anis alcohol or cinnamyl alcohol, secondary monoamines such as dimethylamine, diethylamine, dipropylamine, diisopropylamine, dibutylamine, bis(2-ethylhexyl)amine, N-methylcyclohexylamine, N-ethylcyclohexylamine or dicyclohexylamine and also heterocyclic secondary amines such as morpholine, pyrrolidine, piperidine or 1H pyrazole. Preferred starter molecules are saturated monoalcohols of the aforementioned kind. Particular preference is given to using diethylene glycol monobutyl ether or n-butanol as starter molecules.

Useful alkylene oxides for the alkoxylation reaction are in particular ethylene oxide and propylene oxide, which can be used in any desired order or else in admixture in the alkoxylation reaction.

In a further embodiment of the layered composite of the invention, the free NCO groups of the prepolymers are further reacted in whole or in part in step B) with
B2) amino-functional compounds having molecular weights of ≧32 g/mol to ≦400 g/mol.

Component B2) may utilize di- or polyamines such as 1,2-ethylenediamine, 1,2-diaminopropane, 1,3-diaminopropane, 1,4-diaminobutane, 1,6-diaminohexane, isophoronediamine, isomeric mixtures of 2,2,4- and 2,4,4-trimethylhexamethylenediamine, 2-methylpentamethylenediamine, diethylenetriamine, triaminononane, 1,3-xylylenediamine, 1,4-xylylenediamine, α,α,α′,α′-tetramethyl-1,3- and -1,4-xylylenediamine and 4,4-diaminodicyclohexylmethane and/or dimethylethylenediamine. It is also possible but less preferable to use hydrazine and also hydrazides such as adipohydrazide.

Component B2) can further utilize compounds which as well as a primary amino group also have secondary amino groups or which as well as an amino group (primary or secondary) also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Component B2) can further utilize monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide-amines formed from diprimary amines and monocarboxylic acids, monoketimes of diprimary amines, primary/tertiary amines, such as N,N-dimethylaminopropylamine. Preferred compounds for component B2) are 1,2-ethylenediamine, 1,4-diaminobutane and isophoronediamine

In a further embodiment of the layered composite of the invention, in the preparation of the aqueous, anionically hydrophilicized polyurethane dispersions (I), the component A1) is selected from the group comprising 1,6-hexamethylene diisocyanate, isophorone diisocyanate and/or the isomeric bis-(4,4′-isocyanatocyclohexyl)methanes. The component A2) furthermore comprises a mixture of polycarbonate polyols and polytetramethylene glycol polyols, wherein the proportion of component A2) which is accounted for by the sum total of the polycarbonate polyols and the polytetramethylene glycol the polyether polyols is ≧70% by weight to ≦100% by weight.

In addition to the polyurethane dispersions (I) and the admixtures, it is also possible to use further auxiliary materials.

Examples of such auxiliary materials are thickeners/thixotropine agents, antioxidants, photostabilizers, emulsifiers, plasticizers, pigments, fillers and/or flow control agents.

Commercially available thickeners can be used, such as derivatives of dextrin, of starch or of cellulose, examples being cellulose ethers or hydroxyethylcellulose, organic wholly synthetic thickeners based on polyacrylic acids, polyvinylpyrrolidones, poly(meth)acrylic compounds or polyurethanes (associative thickeners) and also inorganic thickeners such as bentonites or silicas.

In principle, the compositions of the invention can also contain crosslinkers such as unblocked polyisocyanates, amide- and amine-formaldehyde resins, phenolic resins, aldehydic and ketonic resins, examples being phenol-formaldehyde resins, resols, furan resins, urea resins, carbamic ester resins, triazine resins, melamine resins, benzoguanamine resins, cyanamide resins or aniline resins.

In a further embodiment of the layered composite of the invention, the material of the absorbent layer comprises a copolymer of acrylic acid and sodium acrylate or a crosslinked copolymer of acrylic acids with bi- and/or polyfunctional monomers. Examples of bi- and/or polyfunctional monomers are polyallyl glucoses. It is possible here for the absorbent layer to be present in the form of a nonwoven, a powder and/or a granulate. A nonwoven is preferably used for the absorbent layer.

In a further embodiment of the layered composite of the invention, the material of the covering layer comprises the same polyurethane foam as is present in the base layer. This reduces manufacturing costs, since no additional material has to be provided for the covering layer. The covering layer may be applied as an aqueous foam and then dried. It is further possible to ensure strong adherence between the base layer and the covering layer when they are made of the same material.

In a further embodiment of the layered composite of the invention, the direct bond between the base layer and the covering layer has a peel strength of ≧0.8 N/mm. Maximum peel strength can be for example ≦5 N/mm, ≦4 N/mm or ≦3 N/mm. Peel strength can be determined on a Zwick universal tester. In such a peel strength test, the base layer and the covering layer were peeled off each other at an angle of 180° at a traverse speed of 100 mm/min. In those cases where the strength of the bond is greater than the strength of the foam as such, a peel strength of ≧0.8 N/mm was found for the advancing crack in the foam and hence for the lower limit of the strength of the bond.

In a further embodiment of the layered composite of the invention, the water vapour permeability of the covering layer is in the range from ≧750 g/m²/24 hours to ≦5000 g/m²/24 hours. Water vapour permeability can also be in the range from ≧1000 g/m²/24 hours to ≦4000 g/m²/24 hours or in the range from ≧1500 g/m²/24 hours to ≦3000 g/m²/24 hours. Water vapour permeability can be determined as described in the standard DIN EN 13726-2 part 3.2.

An exemplary recipe for preparing the polyurethane dispersions utilizes the components A1) to A4) and B1) to B2) in the following amounts, the individual amounts always adding up to ≦100% by weight:

≧5% by weight to ≦40% by weight of component A1);

≧55% by weight to ≦90% by weight of component A2);

≧0.5% by weight to ≦20% by weight of the sum total of components A3) and B2);

≧0.1% by weight to ≦25% by weight of the sum total of components A4) and B1), wherein, based on the total amounts of the components A1) to A4) and B1) to B2), ≧0.1% by weight to ≦5% by weight of anionic or potentially anionic hydrophilicizing agents from A4) and/or B1) are used.

A further exemplary recipe for preparing the polyurethane dispersions utilizes the components A1) to A4) and B1) to B2) in the following amounts, the individual amounts always adding up to ≦100% by weight:

≧5% by weight to ≦35% by weight of component A1);

≧60% by weight to ≦90% by weight of component A2);

≧0.5% by weight to ≦15% by weight of the sum total of components A3) and B2);

≧0.1% by weight to ≦15% by weight of the sum total of components A4) and B1), wherein, based on the total amounts of the components A1) to A4) and B1) to B2), ≧0.2% by weight to ≦4% by weight of anionic or potentially anionic hydrophilicizing agents from A4) and/or B1) are used.

A very particularly preferred recipe for preparing the polyurethane dispersions utilizes the components A1) to A4) and B1) to B2) in the following amounts, the individual amounts always adding up to ≦100% by weight:

≧10% by weight to ≦30% by weight of component A1);

≧65% by weight to ≦85% by weight of component A2);

≧0.5% by weight to ≦14% by weight of the sum total of components A3) and B2);

≧0.1% by weight to ≦13.5% by weight of the sum total of components A4) and B1), wherein, based on the total amounts of the components Al) to A4) and B1) to B2), 0.5% by weight to ≦3.0% by weight of anionic or potentially anionic hydrophilicizing agents from A4) and/or B 1) are used.

The production of the anionically hydrophilicized polyurethane dispersions (I) can be carried out in one or more stages in homogeneous phase or, in the case of a multistage reaction, partly in disperse phase. After completely or partially conducted polyaddition from A1) to A4) a dispersing, emulsifying or dissolving step is carried out. This is followed if appropriate by a further polyaddition or modification in disperse phase.

Processes such as for example the prepolymer mixing process, the acetone process or the melt dispersing process can be used. The acetone process is preferred.

Production by the acetone process typically involves the constituents A2) to A4) and the polyisocyanate component A1) being wholly or partly introduced as an initial charge to produce an isocyanate-functional polyurethane prepolymer and optionally diluted with a water-miscible but isocyanate-inert solvent and heated to temperatures in the range from ≧50 to ≦120° C. The isocyanate addition reaction can be speeded using the catalysts known in polyurethane chemistry.

Useful solvents include the customary aliphatic, keto-functional solvents such as acetone, 2-butanone, which can be added not just at the start of the production process but also later, optionally in portions. Acetone and 2-butanone are preferred.

Other solvents such as xylene, toluene, cyclohexane, butyl acetate, methoxypropyl acetate, N-methylpyrrolidone, N-ethylpyrrolidone, solvents having ether or ester units can additionally be used and wholly or partly distilled off or in the case of N-methylpyrrolidone, N-ethylpyrrolidone remain completely in the dispersion. But preference is given to not using any other solvents apart from the customary aliphatic, keto-functional solvents. ≧Subsequently, any constituents of A1) to A4) not added at the start of the reaction are added.

In the production of the polyurethane prepolymer from A1) to A4), the amount of substance ratio of isocyanate groups to with isocyanate-reactive groups is for example in the range from ≧1.05 to ≦3.5, preferably in the range from ≧1.2 to ≦3.0 and more preferably in the range from ≧1.3 to ≦2.5.

The reaction of components A1) to A4) to form the prepolymer is effected partially or completely, but preferably completely. Polyurethane prepolymers containing free isocyanate groups are obtained in this way, without a solvent or in solution.

The neutralizing step to effect partial or complete conversion of potentially anionic groups into anionic groups utilizes bases such as tertiary amines, for example trialkylamines having ≧1 to ≦12 and preferably ≧1 to ≦6 carbon atoms and more preferably ≧2 to ≦3 carbon atoms in every alkyl radical or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may also bear for example hydroxyl groups, as in the case of the dialkylmonoalkanol-, alkyldialkanol- and trialkanolamines. Useful neutralizing agents further include if appropriate inorganic bases, such as aqueous ammonia solution, sodium hydroxide or potassium hydroxide.

Preference is given to ammonia, triethylamine, triethanolamine, dimethylethanolamine or diisopropylethylamine and also sodium hydroxide and potassium hydroxide, particular preference being given to sodium hydroxide and potassium hydroxide.

The bases are employed in an amount of substance which is between ≧50 and ≦125 mol % and preferably between ≧70 and ≦100 mol % of the amount of substance of the acid groups to be neutralized. Neutralization can also be effected at the same time as the dispersing step, by including the neutralizing agent in the water of dispersion.

Subsequently, in a further process step, if this has not already been done or only to some extent, the prepolymer obtained is dissolved with the aid of aliphatic ketones such as acetone or 2-butanone.

In the chain extension of stage B), NH₂- and/or NH-functional components are reacted, partially or completely, with the still remaining isocyanate groups of the prepolymer. Preferably, the chain extension is carried out before dispersion in water.

Chain termination is typically carried out using amines B2) having an isocyanate-reactive group such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine or suitable substituted derivatives thereof, amide-amines formed from diprimary amines and monocarboxylic acids, monoketimes of diprimary amines, primary/tertiary amines, such as N,N-dimethylaminopropylamine.

When partial or complete chain extension is carried out using anionic or potentially anionic hydrophilicizing agents conforming to definition B1) with NH₂ or NH groups, chain extension of the prepolymers is preferably carried out before dispersion.

The aminic components B1) and B2) can optionally be used in water- or solvent-diluted form in the process of the invention, individually or in mixtures, any order of addition being possible in principle.

When water or organic solvent is used as a diluent, the diluent content of the chain-extending component used in B) is preferably in the range from ≧70% to ≦95% by weight.

Dispersion is preferably carried out following chain extension. For dispersion, the dissolved and chain-extended polyurethane polymer is either introduced into the dispersing water, if appropriate by substantial shearing, such as vigorous stirring for example, or conversely the dispersing water is stirred into the chain-extended polyurethane polymer solutions. It is preferable to add the water to the dissolved chain-extended polyurethane polymer.

The solvent still present in the dispersions after the dispersing step is then typically removed by distillation. Removal during the dispersing step is likewise possible.

The residual level of organic solvents in the polyurethane dispersions (I) is typically less than ≦1.0% by weight and preferably less than ≦0.5% by weight, based on the entire dispersion.

The pH of the polyurethane dispersions (I) of the present invention is typically ≦9.0, preferably ≦8.5, more preferably ≦8.0 and most preferably is in the range from ≧6.0 to ≦7.5.

The solids content of the polyurethane dispersions (I) is preferably in the range from ≧40% to ≦70% by weight, more preferably in the range from ≧50% to ≦65% by weight, even more preferably in the range from ≧55% to ≦65% by weight and in particular in the range from ≧60% to ≦65% by weight.

Examples of compositions according to the invention are recited hereinbelow, the sum total of the weights in % ages has a value of ≦100% by weight. These compositions, based on dry substance, typically comprise ≧80 parts by weight to ≦99.5 parts by weight of dispersion (I), ≧0 parts by weight to ≦10 parts by weight of foam auxiliary, ≧0 parts by weight to ≦10 parts by weight of crosslinker and ≧0 parts by weight to ≦10 parts by weight of thickener.

These compositions according to the invention, based on dry substance, preferably comprise ≧85 parts by weight to ≦97 parts by weight of dispersion (I), ≧0.5 parts by weight to ≦7 parts by weight of foam auxiliary, ≧0 parts by weight to ≦5 parts by weight of crosslinker and ≧0 parts by weight to ≦5 parts by weight of thickener.

These compositions according to the invention, based on dry substance, more preferably comprise ≧89 parts by weight to ≦97 parts by weight of dispersion (I), ≧0.5 parts by weight to ≦6 parts by weight of foam auxiliary, ≧0 parts by weight to ≦4 parts by weight of crosslinker and ≧0 parts by weight to ≦4 parts by weight of thickener.

Examples of compositions according to the invention which comprise ethylene oxide/propylene oxide block copolymers as foam stabilizers are recited hereinbelow. These compositions, based on dry substance, comprise ≧80 parts by weight to ≦99.9 parts by weight of dispersion (I) and ≧0.1 parts by weight to ≦20 parts by weight of the ethylene oxide/propylene oxide block copolymers. The compositions, based on dry substance, preferably comprise ≧85 parts by weight to ≦99.5 parts by weight of dispersion (I) and 0.5 to 15 parts by weight of the ethylene oxide/propylene oxide block copolymers. Particular preference here is given to ≧90 parts by weight to ≦99 parts by weight of dispersion (I) and ≧1 part by weight to ≦10 parts by weight of the ethylene oxide/propylene oxide block copolymers and very particular preference is given to ≧94 parts by weight to ≦99 parts by weight of dispersion (I) and ≧1 to ≦6 parts by weight of the ethylene oxide/propylene oxide block copolymers.

For the purposes of the present invention, “parts by weight” denotes a relative proportion, but not in the sense of % by weight. Consequently, the arithmetic sum total of the proportions by weight can also assume values above 100.

In addition to the components mentioned, the compositions according to the invention may also utilize further aqueous binders. Such aqueous binders can be constructed for example of polyester, polyacrylate, polyepoxy or other polyurethane polymers. Similarly, the combination with radiation-curable binders as described for example in EP-A-0 753 531 is also possible. It is further possible to employ other anionic or nonionic dispersions, such as polyvinyl acetate, polyethylene, polystyrene, polybutadiene, polyvinyl chloride, polyacrylate and copolymer dispersions.

Frothing in the process of the present invention is accomplished by mechanical stirring of the composition at high speeds of rotation by shaking or by decompressing a blowing gas.

Mechanical frothing can be effected using any desired mechanical stirring, mixing and dispersing techniques. Air is generally introduced, but nitrogen and other gases can also be used for this purpose.

The invention further provides a process for producing a layered composite according to the present invention, comprising the steps of
providing a base layer comprising a polyurethane foam obtained by a composition comprising an aqueous, anionically hydrophilicized polyurethane dispersion (I) being frothed and dried;
applying an absorbent layer atop the base layer;
applying a further layer so that this further layer is bonded both to the base layer and to the absorbent layer.

The application of an absorbent layer atop the base layer can be effected in the case of a superabsorbent powder or granulate by simply sprinkling with or without assistance of a template. In the case of a superabsorbent nonwoven, this nonwoven can be placed on the base layer in the form of suitably cut pads, mechanically or by hand. The use of a superabsorbent nonwoven is preferred.

Conceivable embodiments of the process according to the invention include inter alia the variants described hereinbelow. One variant comprises drying the base layer, providing it with the absorbent layer, applying the covering layer and drying again.

In another variant, the absorbent/superabsorbent is applied to the undried base layer, the composite material obtained is dried, subsequently overcoated with a covering layer and dried again.

In another variant, the absorbent layer is applied to a substrate such as paper or foil, film or sheet, the base layer or covering layer is applied atop the absorbent layer, the composite material obtained is dried, subsequently the layer which is still missing (covering layer or base layer) is applied atop the composite material on the absorbent side, and another drying step is carried out.

In another variant, the absorbent layer is applied atop a substrate such as paper or foil, film or sheet, the base layer or covering layer is applied, the composite material is dried and is placed with its absorbent side on the still missing layer (covering layer or base layer), and another drying step is carried out.

In another variant, the absorbent/superabsorbent is applied to the undried base layer, overcoated with a covering layer and the composite material is dried in a single operation.

A preferred variant comprises drying the base layer, providing absorbent layer, applying the covering layer and drying again.

The foam obtained is, in the course of frothing or immediately thereafter, applied atop a substrate or introduced into a mould and dried. Useful substrates include in particular papers, foils, films or sheets, which permit simple peeling off of the wound dressing before its use for covering an injured site.

Application can be for example by pouring or blade coating, but other conventional techniques are also possible. Multilayered application with intervening drying steps is also possible in principle.

A satisfactory drying rate for the foams is observed at a temperature as low as 20° C., so that drying on injured human or animal tissue presents no problem. However, temperatures above 30° C. are preferably used for more rapid drying and fixing of the foams. However, drying temperatures should not exceed 200° C., preferably 180° C. and more preferably 150° C., since undesirable yellowing and/or liquefication of the foams can otherwise occur. Drying in two or more stages is also possible.

Drying is generally effected using conventional heating and drying apparatus, such as (circulating air) drying cabinets, hot air or IR radiators. Drying by leading the coated substrate over heated surfaces, for example rolls, is also possible.

Application and drying can each be carried out batchwise or continuously, but an entirely continuous process is preferred.

Before drying, the foam densities of the polyurethane foams are typically in an range from ≧50 g/litre to ≦800 g/litre, preferably ≧100 g/litre to ≦500 g/litre and more preferably ≧100 g/litre to ≦350 g/litre (mass of all input materials [in g] based on the foam volume of one litre).

After drying, the polyurethane foams can have a microporous, at least partially open-pore structure having intercommunicating cells. The density of the dried foams is typically below 0.4 g/cm³, preferably below 0.35 g/cm³, more preferably in the range from ≧0.01 g/cm³ to ≦0.3 g/cm³ and most preferably in the range from ≧0.1 g/cm³ to ≦0.3 g/cm³.

After drying, the thickness of the polyurethane foam layers is typically in the range from ≧0.1 mm to ≦50 mm, preferably ≧0.5 mm to ≦20 mm, more preferably ≧1 mm to ≦10 mm and most preferably ≧1.5 mm to ≦5 mm.

One embodiment of the process according to the invention comprises the further layer being obtained by a composition comprising an aqueous, anionically hydrophilicized polyurethane dispersion (I) being frothed and wherein, after application of the further layer, the layered composite is dried. Drying can take place for example at a temperature of ≧110° C. to ≦150° C. This gives a layered composite where the base layer and the covering layer preferably comprise the same material.

The present invention further provides for the use of a layered composite according to the present invention as wound dressing, incontinence product and/or cosmetic article. Incontinence products can be for example diapers for babies, children and adults. Cosmetic articles can be cleaning articles for example. The use as wound dressing is preferred.

The present invention is further elucidated with reference to the following drawing, where

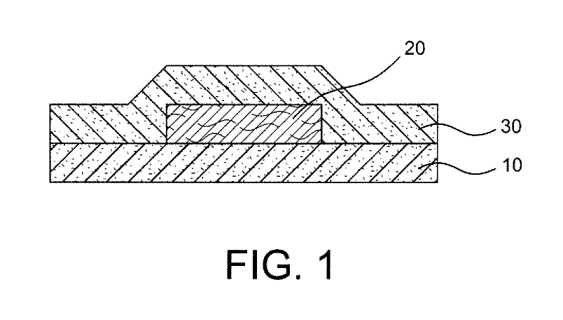

FIG. 1 shows a cross-sectional view of an inventive layered composite.

FIG. 1 shows a cross-sectional view of an inventive layered composite. The base layer 10 is embodied as a polyurethane foam layer, the polyurethane foam being obtainable as described. Atop the base layer 10 is the absorbent layer 20. In the present illustrative embodiment, the absorbent layer 20 is constituted by a textile superabsorbent, for example in the form of a nonwoven. The covering layer 30 in the present case is likewise embodied as a polyurethane foam layer, the polyurethane foam being obtainable as described. The covering layer 30 covers not only the absorbent layer 20 but also the base layer 10, so that an island dressing is obtained.

The present invention is further elucidated with reference to the examples which follow.

Unless indicated otherwise, all percentages are by weight.

Solids contents were determined in accordance with DIN-EN ISO 3251. NCO contents were, unless expressly mentioned otherwise, determined volumetrically in accordance with DIN-EN ISO 11909. “Free absorbency” was determined by absorption of physiological saline in accordance with DIN EN 13726-1 Part 3.2.

Substances and Abbreviations Used:

| | |
|---|---|
| diaminosulphonate: | NH₂—CH₂CH₂—NH—CH₂CH₂—SO₃Na (45% in water) |
| Desmophen ® C2200: | polycarbonate polyol, OH number 56 mg KOH/g, number average |
| | molecular weight 2000 g/mol (Bayer MaterialScience AG, |
| | Leverkusen, Germany) |
| PolyTHF ® 2000: | polytetramethylene glycol polyol, OH number 56 mg KOH/g, |
| | number average molecular weight 2000 g/mol (BASF AG, |
| | Ludwigshafen, Germany) |
| PolyTHF ® 1000: | polytetramethylene glycol polyol, OH number 112 mg KOH/g, |
| | number average molecular weight 1000 g/mol (BASF AG, |
| | Ludwigshafen, Germany) |
| LB 25 polyether: | monofunctional polyether based on ethylene oxide/propylene |
| | oxide, number average molecular weight 2250 g/mol, OH number |
| | 25 mg KOH/g (Bayer MaterialScience AG, Leverkusen, Germany) |
| Plantacare ® 1200 UP: | C₁₂-C₁₆ fatty alcohol-polyglycoside, about 51% solution in water |
| | (Cognis Deutschland GmbH & Co. KG, Düsseldorf, Germany) |
| Stokal ® STA: | ammonium stearate, about 30% solution in water (Bozzetto GmbH, |
| | Krefeld, Germany) |
| Pluronic ® PE 6800: | EO/PO block copolymer, weight average molecular weight |
| | 8000 g/mol (BASF AG, Ludwigshafen, Germany) |
| OASIS SAF ® 2342: | nonwoven superabsorbent based on acrylic acid, methacrylic acid |
| | and an acrylic acid/methacrylic acid monomer in which the acrylic |
| | acid was partially neutralized to sodium acrylate. The crosslinks |
| | between the polymer chains are obtained by means of ester groups |
| | from the reaction between acid groups of the acrylic acid and |
| | hydroxyl groups in the acrylic acid/methacrylic acid monomer |
| | (Technical Absorbents Ltd., UK). |
| OASIS SAF ® 2317: | nonwoven superabsorbent based on acrylic acid, methacrylic acid |
| | and an acrylic acid/methacrylic acid monomer in which the acrylic |
| | acid was partially neutralized to sodium acrylate. The crosslinks |
| | between the polymer chains are obtained by means of ester groups |
| | from the reaction between acid groups of the acrylic acid and |
| | hydroxyl groups in the acrylic acid/methacrylic acid monomer |
| | (Technical Absorbents Ltd., UK). |
| OASIS SAF ® 2354: | nonwoven superabsorbent based on acrylic acid, methacrylic acid |
| | and an acrylic acid/methacrylic acid monomer in which the acrylic |
| | acid was partially neutralized to sodium acrylate. The crosslinks |
| | between the polymer chains are obtained by means of ester groups |
| | from the reaction between acid groups of the acrylic acid and |
| | hydroxyl groups in the acrylic acid/methacrylic acid monomer |
| | (Technical Absorbents Ltd., UK). |
| Favor ® PAC 230: | pulverulent superabsorbent based on crosslinked polyacrylate |
| | (Evonik Stockhausen GmbH, Krefeld, Germany) |
| Luquafleece ® 200: | nonwoven superabsorbent based on crosslinked polyacrylate |
| | (BASF AG, Ludwigshafen, Germany) |
| Luquafleece ® 400: | nonwoven superabsorbent based on crosslinked polyacrylate |
| | (BASF AG, Ludwigshafen, Germany) |

The determination of the average particle sizes (the number average is reported) of polyurethane dispersion 1 was carried out using laser correlation spectroscopy (LCS; instrument: Malvern Zetasizer 1000, Malver Inst. Limited).

The contents reported for the foam additives are based on aqueous solutions.

### Example 1

### Production of Polyurethane Dispersion 1

1077.2 g of PolyTHF® 2000, 409.7 g of PolyTHF® 1000, 830.9 g of Desmophen® C2200 and 48.3 g of LB 25 polyether were heated to 70° C. in a standard stirring apparatus. Then, a mixture of 258.7 g of hexamethylene diisocyanate and 341.9 g of isophorone diisocyanate was added at 70° C. in the course of 5 min and the mixture was stirred at 120° C. until the theoretical NCO value was reached or the actual NCO value was slightly below the theoretical NCO value. The ready-produced prepolymer was dissolved with 4840 g of acetone and, in the process, cooled down to 50° C. and subsequently admixed with a solution of 27.4 g of ethylenediamine, 127.1 g of isophoronediamine, 67.3 g of diaminosulphonate and 1200 g of water metered in over 10 min. The mixture was subsequently stirred for 10 min. Then, a dispersion was formed by addition of 654 g of water. This was followed by removal of the solvent by distillation under reduced pressure.

The polyurethane dispersion obtained had the following properties:

| | |
|---|---|
| Solids content: | 61.6% |
| Particle size (LCS): | 528 nm |
| pH (23° C.): | 7.5 |

### Example 2

### Production of a Foam-Superabsorbent Composite Material from Polyurethane Dispersion 1

120 g of polyurethane dispersion 1, produced according to Example 1, were mixed with 1.47 g of Plantacare® 1200 UP and 0.24 g of Stokal® STA and frothed by means of a commercially available hand stirrer (stirrer made of bent wire) to a 0.4 litre foam volume. Thereafter, the foam was drawn down on non-stick paper by means of a blade coater set to a gap height of 2 mm, subsequently, an approximately 5*5 cm² nonwoven of a superabsorbent (see Table 1) was laid without pressure onto the still moist foam and dried for 15 minutes at 120° C. in a circulating air drying cabinet. Then, a further layer of foam was drawn down, by means of a film coater set to a gap height of 6 mm, over the previously dried foam-superabsorbent composite material such that the superabsorbent nonwoven was completely enclosed by the two layers of foam: by the already dried layer of foam underneath and by the still moist layer of foam at the top and along the sides. The composite material was dried again at 120° C. for 20 minutes in a circulating air drying cabinet.

Clean white foam-superabsorbent composite materials having good mechanical properties and a fine porous structure were obtained.

TABLE 1

| Superabsorbent | Absorbency of composite material |
|---|---|
| OASIS type 2342 | not determined |
| OASIS type 2317 | 95 g/100 cm² |
| OASIS type 2354 | 63 g/100 cm² |

To determine absorbency in accordance with DIN EN 13726-1 Part 3.2, test specimens of 5 cm×5 cm edge length were cut out of each composite material. These test specimens contained 5 cm×5 cm of the absorbent layer.

### Example 3

### Production of a Foam-Superabsorbent Composite Material from Polyurethane Dispersion 1

In a manner analogous to Example 2, a frothed foam was produced from 120 g of polyurethane dispersion 1, from 1.47 g of Plantacare® 1200 UP and 0.24 g of Stokal® STA and was drawn down on non-stick paper by means of a blade coater set to a gap height of 2 mm. Favor® PAC 230 pulverulent superabsorbent was sprinkled onto the still moist foam in the form of a 5 cm×5 cm square. This was followed by drying for 15 minutes at 120° C. in a circulating air drying cabinet. Then, a further layer of the polyurethane foam was drawn down, by means of a blade coater set to a gap height of 4 mm, over the previously dried foam-superabsorbent composite material, such that the superabsorbent was completely enclosed. The composite material was dried again at 120° C. for 20 minutes in a circulating air drying cabinet.

A clean white foam-superabsorbent composite material having good mechanical properties, high absorbency and a fine porous structure was obtained.

### Example 4

### Production of a Foam-Superabsorbent Composite Material from Polyurethane Dispersion 1 and Ethylene Oxide/Propylene Oxide Block Copolymers

120 g of polyurethane dispersion 1, produced according to Example 1, were mixed with 12.6 g of a 30% solution of Pluronic® PE 6800 in water and frothed by means of a commercially available hand stirrer (stirrer made of bent wire) to a 0.4 litre foam volume. Thereafter, the foam was drawn down on non-stick paper by means of a blade coater set to a gap height of 2 mm, subsequently, an approximately 5 cm×5 cm nonwoven of a superabsorbent (see Table 2) was laid without pressure onto the still moist foam and dried for 10 minutes at 120° C. in a circulating air drying cabinet.

Then, a further layer of foam was drawn down on non-stick paper by means of a blade coater again set to a gap height of 2 mm, and the previously dried foam-superabsorbent nonwoven was placed atop the still moist foam such that the superabsorbent nonwoven was enclosed on both sides by polyurethane foam. The composite material was dried again at 120° C. for 10 minutes in a circulating air drying cabinet.

Clean white foam-superabsorbent composite materials having good mechanical properties (peel strength ≧0.8 N/mm) and a fine porous structure were obtained.

TABLE 2

| Superabsorbent | Absorbency of composite material |
|---|---|
| Luquafleece 200 | 103 g/100 cm² |
| Luquafleece 400 | 146 g/100 cm² |

In a departure from DIN EN 13726-1 Part 3.2, absorbency was determined using in each case layered composites having an edge length of 8.5 cm×8.5 cm, which contained 5 cm×5 cm of absorbent layer.

1. Layered composite useful as a wound dressing, comprising a base layer, an absorbent layer atop the base layer, and also a covering layer, wherein the covering layer is bonded to both the base layer and the absorbent layer and wherein the base layer comprises a polyurethane foam obtained by a composition comprising an aqueous, anionically hydrophilicized polyurethane dispersion that has been frothed and dried.

2. Layered composite according to claim 1, wherein the composition from which the polyurethane foam of the base layer is obtained further comprises at least one selected from the group consisting of fatty acid amides, sulphosuccinamides, hydrocarbonsulphonates, hydrocarbyl sulphates, fatty acid salts, alkylpolyglycosides and ethylene oxide-propylene oxide block copolymers.

3. Layered composite according to claim 2, wherein the ethylene oxide-propylene oxide block copolymers have a structure conforming to formula (1): 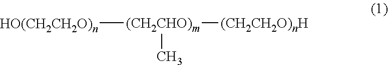
where n is in the range from ≧2 to ≦200 and m is in the range from ≧10 to ≦60.

4. Layered composite according to claim 1, wherein the aqueous, anionically hydrophilicized polyurethane dispersion is obtainable by
A) providing at least one isocyanate-functional prepolymer obtainable from a reaction mixture comprising
A1) at least one organic polyisocyanate and
A2) at least one polymeric polyol having a number average molecular weight of ≧400 g/mol to ≦8000 g/mol and an OH functionality of ≧1.5 to ≦6 and subsequently
B) reacting free NCO groups of the prepolymer in whole or in part with
B1) at least one isocyanate-reactive anionic or potentially anionic hydrophilicizing agent
with chain extension and dispersing the prepolymer in water before, during or after step B), wherein potentially anionic groups still present in the reaction mixture are converted into their ionic form by partial or complete reaction with a neutralizing agent.

5. Layered composite according to claim 4, wherein the reaction mixture in step A) further comprises:
A3) at least one hydroxyl-functional compound having a molecular weight of ≧62 g/mol to ≦399 g/mol.

6. Layered composite according to claim 4, wherein the reaction mixture in step A) further comprises:
A4) at least one isocyanate-reactive anionic, potentially anionic and/or nonionic hydrophilicizing agent.

7. Layered composite according to claim 4, wherein the free NCO groups of the prepolymer is further reacted in whole or in part in step B) with
B2) at least one amino-functional compound having a molecular weight of ≧32 g/mol to ≦400 g/mol.

8. Layered composite according to claim 4, wherein, in the preparation of the aqueous, anionically hydrophilicized polyurethane dispersions, the component A1) is at least one selected from the group consisting of 1,6-hexamethylene diisocyanate, isophorone diisocyanate and the isomeric bis-(4,4′-isocyanatocyclohexyl)methanes and wherein furthermore the component A2) comprises a mixture of at least one polycarbonate polyol and at least one polytetramethylene glycol polyol, wherein the proportion of component A2) which is accounted for by the sum total of the polycarbonate polyol and the polytetramethylene glycol polyether polyol is ≧70% by weight to ≦100% by weight.

9. Layered composite according to claim 1, wherein the material of the absorbent layer comprises a copolymer of acrylic acid and sodium acrylate or a crosslinked copolymer of acrylic acid with at least one bi- and/or polyfunctional monomer.

10. Layered composite according to claim 1, wherein the material of the covering layer comprises the same polyurethane foam as present in base layer.

11. Layered composite according to claim 1, wherein a direct bond between the base layer and the covering layer has a peel strength of ≧0.8 N/mm.

12. Layered composite according to claim 1, wherein the water vapour permeability of the covering layer is in the range from ≧750 g/m²/24 hours to ≦5000 g/m²/24 hours.

13. Process for producing a layered composite according to claim 1, comprising the steps of
providing a base layer comprising a polyurethane foam obtained by a composition comprising an aqueous, anionically hydrophilicized polyurethane dispersion that has been frothed and dried;
applying an absorbent layer atop the base layer;
applying a further layer so that said further layer is bonded both to the base layer and to the absorbent layer.

14. Process according to claim 13, wherein the further layer is obtained by a composition comprising an aqueous, anionically hydrophilicized polyurethane dispersion that has been frothed and wherein, after application of the further layer, the layered composite is dried.

15. A wound dressing, incontinence product and/or cosmetic article comprising a layered composite of claim 1.
